(19) United States
(12) Patent Application Publication
(10) Pub. No.: US 2010/0004748 A1
(43) Pub. Date: Jan. 7, 2010

(54) INTERVERTEBRAL PROSTHESIS

(76) Inventor: Nicholas M. Cordaro, Vista, CA (US)

Correspondence Address: SNELL & WILMER LLP (OC), 600 ANTON BOULEVARD, SUITE 1400, COSTA MESA, CA, 92626, US

(21) Appl. No.: 12/496,582

(22) Filed: Jul. 1, 2009

Related U.S. Application Data
(60) Provisional application No. 61/133,865, filed on Jul. 3, 2008.

(51) Int. Cl. A61F 2/44

(52) U.S. Cl. 623/17.16; 623/17.13

(57) ABSTRACT

An intervertebral prosthesis including an elastically deformable disc arranged around an axis with upper and lower surfaces, oriented generally perpendicular to the axis, for engaging the faces of adjacent vertebral bodies between which a natural disc has been wholly or partially removed. The disc can have an exterior wall and be arranged to undergo flexion, compression and expansion in response to forces applied by the supporting vertebra to the disc. A pin can be provided to limit movement of the disc. A portion of the disc such as the central section can have one or more generally horizontal slits extending perpendicular to the axis to provide a spring function.

### CROSS-REFERENCE TO RELATED APPLICATION

This application claims priority from Provisional Application No. 61/133,865 filed on Jul. 3, 2008.

### BACKGROUND OF THE INVENTION

1. Field of the Invention

This application relates to a spinal prosthesis for restoring the motion of adjacent intervertebral bodies between which a natural disc has been wholly or partially removed.

2. Description of Related Art

An earlier patent application, publication number W02007/07541 (“'541 application”), owned by the assignee of this application, discloses a variety of mechanical discs suitable for replacing a failed natural disc while restoring, to a large degree, the natural motion between the supported vertebrae and in particular, discs that promote soft fusion as that term is defined in the application. The contents of the '541 application are incorporated herein by reference.

The discs disclosed in the '541 application, as well as many prior art artificial spinal discs, are designed with upper and lower sections or plates which interface with the faces of the vertebral bodies to be supported. A mechanical compression spring arrangement is interposed between the sections or plates to maintain the desired spacial relationship between the supported vertebrae while allowing normal movement of the vertebrae during the flexion, compression and extension of the spring arrangement. The exterior faces of the discs are typically coated with a bone adhering interface material, such as titanium, or provided with keels and the like for insuring that the patient's bone will strongly adhere to the exposed faces of the disc.

A concern has arisen that while a secure bond between the exterior surfaces of the discs and the underlying vertebrae serves a very important function of maintaining the disc in place, it also has the potential of extending the spring arrangement beyond its elastic limit under repeated movements of the supported vertebrae resulting in a shortened life for the disc. This concern is primarily focused on smaller, less robust discs used, for example, in the cervical or thoracic regions. The present invention is directed to overcoming this problem.

### SUMMARY OF THE INVENTION

An improved intervertebral prosthesis, in accordance with this invention, includes an elastically deformable disc arranged around an imaginary longitudinal axis having upper and lower vertebral body engaging surfaces which are oriented in planes generally perpendicular to the longitudinal axis. The deformable disc is arranged to undergo flexion, compression and expansion in response to forces applied thereto by the supported vertebrae to mimic the response of a natural disc. To overcome a possible anticipated premature fatigue problem, the disc can include at least one expansion or extension limit pin extending therethrough. Device flexion being device movement in any combinations of flexion/extension or lateral bending movements.

In one embodiment the disc is formed with a plurality of interleaved slots with alternate slots extending partially through the disc from opposing sides thereof. The slots are oriented in a direction generally perpendicular to the longitudinal axis with the depths of the slots decreasing and increasing during the compression and expansion of the overlying portion of the disc, respectively. Optionally, the disc may define one or more longitudinally oriented bone channels extending through the disc for accommodating bone growth therein.

In another embodiment, the disc may have an oval shape with top and bottom plates separated by an outer helical spring, the outside surfaces of the plates serving to engage the faces of the separated vertebral bodies.

Optionally the device may be elliptical, circular or other geometric shape to accommodate the vertebral bodies and surgical approach.

Optionally the outer helical spring may be rigidly connected to only one first plate serving to engage one separated vertebral body. The opposite plate, serving to engage the opposite separated vertebral body will be coupled to the first plate by at least one expansion or extension limit pin.

Optionally an inner helical spring may be positioned inside of the outer spring and between the plates. The springs may consist of 1 to 2 turns and the top of the outer spring may be formed integrally with the top plate and the bottom of the inner spring may be formed integrally with the bottom plate.

### BRIEF DESCRIPTION OF THE DRAWINGS

The objects and features of the present invention, which are believed to be novel, are set forth with particularity in the appended claims. The present invention, both as to its organization and manner of operation, together with further objects and advantages, may best be understood by reference to the following description, taken in connection with the accompanying drawings.

FIG. 1 is a perspective view of an intervertebral prosthetic disc, in accordance with this invention;

FIG. 2 is intervertebral discs with expansion limit pins in place;

FIG. 17 is a perspective view of another embodiment of an expansion limited intervertebral disc designed for anterior implacement;

FIG. 18 is a cross-sectional view of the disc of FIG. 17;

FIG. 19 is a blown up side elevational view of the unassembled disc of FIG. 17 illustrating the several components thereof;

FIG. 20 is a top plan view of one of the expansion limit pins;

FIGS. 21 and 22 are plan views of the bottom and top spring sections looking along lines 20 and 21 of FIG. 19, respectively;

FIG. 23 is a side elevational view of the assembled disc;

FIGS. 24 and 25 are bottom and top plan views of the disc, respectively; and

FIGS. 26 and 27 are top plan and a side elevational view of an internal spacer for the disc.

### DETAILED DESCRIPTION OF THE PREFERRED EMBODIMENTS

Reference will now be made in detail to the preferred embodiments of the invention which set forth the best modes contemplated to carry out the invention, examples of which are illustrated in the accompanying drawings. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention as defined by the appended claims. Furthermore, in the following detailed description of the present invention, numerous specific details are set forth in order to provide a thorough understanding of the present invention. However, it will be obvious to one of ordinary skill in the art that the present invention may be practiced without these specific details. In other instances, well known methods, procedures and components have not been described in detail as not to unnecessarily obscure aspects of the present invention.

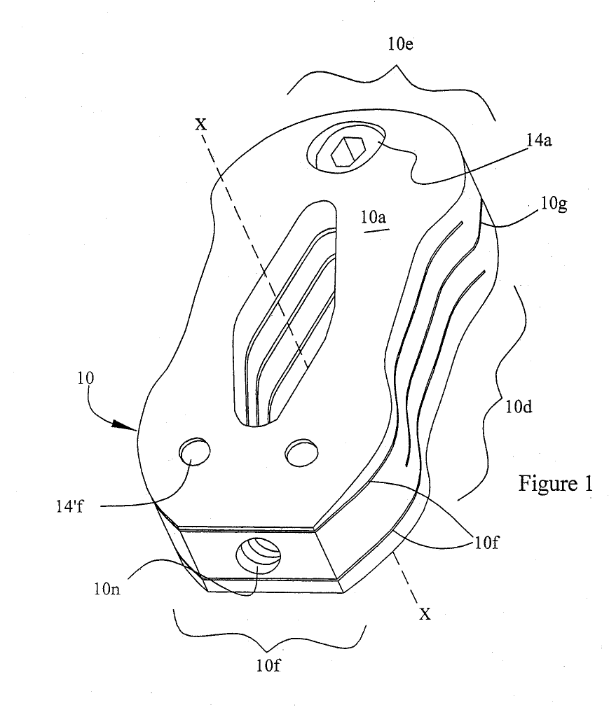

Referring now to FIGS. 1-13, an elastically deformable intervertebral prosthetic disc 10, designed for posterior implantation and formed around an imaginary longitudinal axis x-x, includes upper and lower surfaces 10a and 10b, respectively, adapted to engage the respective faces of adjacent vertebral bodies between which a natural disc has been wholly or partially removed. The upper and lower surfaces would typically be provided with a bone enhancement coating or keels to ensure that the disc will become firmly attached to the underlying vertebral bodies.

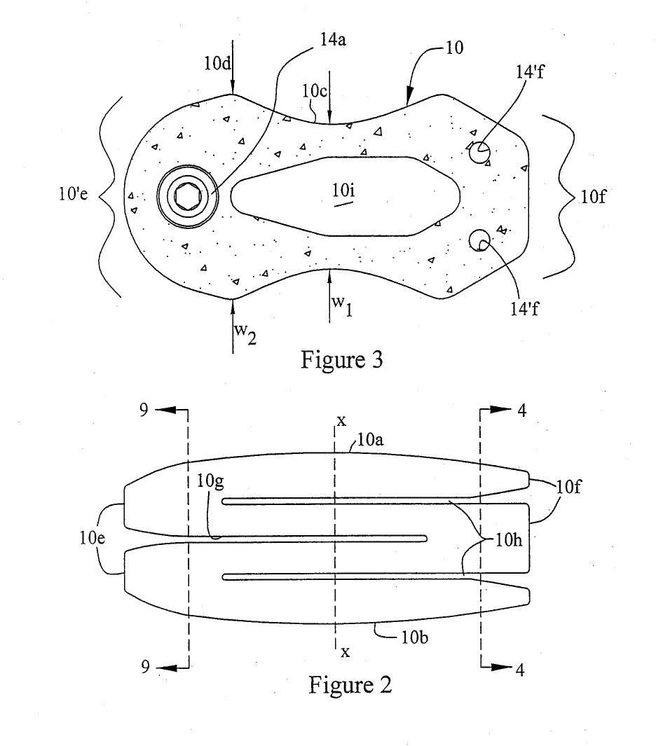

The disc is formed with pinched side walls 10c and expanded or widened intermediate side wall sections 10d adjacent the front and rear end walls 10e and 10f, for accommodating higher loads in the wider intermediate sections and increased bending along the center section. See FIG. 3. The disc is formed with fore and aft horizontally oriented (generally perpendicular to the longitudinal axis) tissue accommodating interleaved channels or slots 10g and 10h, respectively, extending through the front (or nose) and rear (or tail) sections and the side sections as is shown in FIG. 2. The outlet of the slots are tapered as shown to accommodate bending stresses. The slots accommodate the spring action of the disc and the depth thereof decrease and increase during compression and expansion of the relevant portion of the disc. A threaded blind bore 10n is adapted to receive the threaded end of an insertion tool. Examples of the dimensions for such a posterior cervical disc are provided on page 26 of the '541 application. The exterior wall surrounds a vertically oriented bone accommodating channel 10i. It is to be noted that while the term bone channel is used herein to describe a vertically oriented channel for accommodating the infusion of bone therein, the channel may be filled with other biocompatible, compliant and energy absorbing material. See PCT/US2009/39550 filed Apr. 3, 2009 and incorporated herein.

To limit the expansion of the disc and thus provide fatigue protection, one and preferably more, expansion limit pins extend through the disc and are secured thereto at locations above and below the slots. FIG. 4, a cross-sectional view, taken through the distal or nose section 10e, shows a longitudinal pin receiving bore 12 for receiving such a pin. The bore is formed with a lower threaded portion 12a extending into a lower section 10j of the disc which is separated from an upper section 10k by the slot 10g. An expanded diameter unthreaded intermediate portion 12b extends upwardly into the upper section 10k for accommodating the cylindrical shank of a pin 14 to be described in conjunction with FIGS. 5 and 6. The bore terminates in an enlarged pin head accommodating position 12c via an annular shelf 12d.

An expansion limit pin or bolt 14, shown in FIGS. 5, 6, and 7, includes a head 14a which transitions to a shank 14b through an undercut forming an annular face 14c, then through another undercut forming another annular face 14d and then through a reduced diameter neck portion 14e to a lower threaded portion 14f. Pin 14 is formed with a hexagonal wrench engaging depression 14g extending through the head as shown.

The pin 14, when assembled with the disc and in its unstressed condition, is shown in FIG. 8 with the annular face 14d seated against the upper surface 10l of the lower section 10j. The distance d1 between the annular face 14c of the pin and the shelf 12d in the upper section of the disc prescribes the maximum allowable expansion of the disc in the tail region.

FIG. 9, a cross-sectional view, taken through the proximal or tail section 10f (FIG. 2) shows two pin receiving bores 12′b. It is to be noted that features in FIGS. 9-13 which are similar to those discussed in connection with FIGS. 4-8 are identified with prime numerals with the understanding that tail section is formed with two identical limit pin receiving bores 12′ which accommodate two limit pins 14′. The bores 12′ and cooperating pins 14′ are longer than the bores 12 and pins 14 because the greater distance between the upper and lower sections 10k and 10j, separated by the slots 10h and a center section 10m (FIG. 9) located between the slots.

In the assembled, unstressed condition, the pins 14′ are seated on the upper surface 10l of the disc lower section 10j with the annular faces 14′c extending above the shelf 12′d a distance d1 to limit the expansion of the tail section of the disc.

Three additional elastically deformable intervertebral discs are illustrated in FIGS. 14-16. The construction of the discs is similar to that described with respect to the preceding figures with additional interleaved lateral or horizontal slots providing the spring action. FIGS. 14 and 15 are illustrative of a disc designed for anterior implantation while FIG. 16 is illustrative of a disc designed for posterior or lateral implantation. Alternate slots extend from opposite ends to a position near the other end except for the disc of FIG. 16 which includes a centrally located slot extending in each direction from a lateral channel 16a. Expansion limit pins 14″ are inserted through each of the discs and secured to the upper and lower sections (separated by the slots) in the manner illustrated in the preceding figures. Each of the discs include one or more vertically oriented bone growth accommodating channels 10′j as shown.

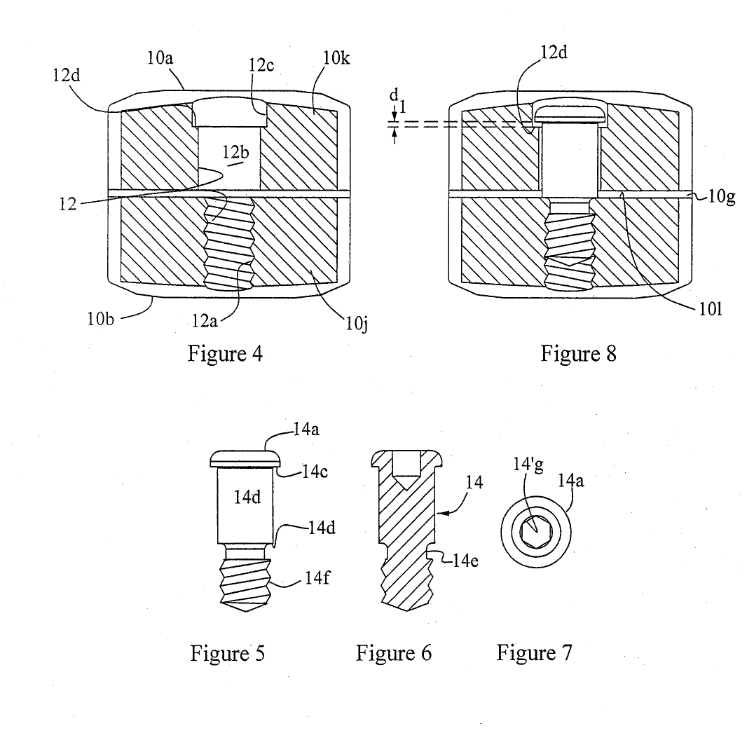

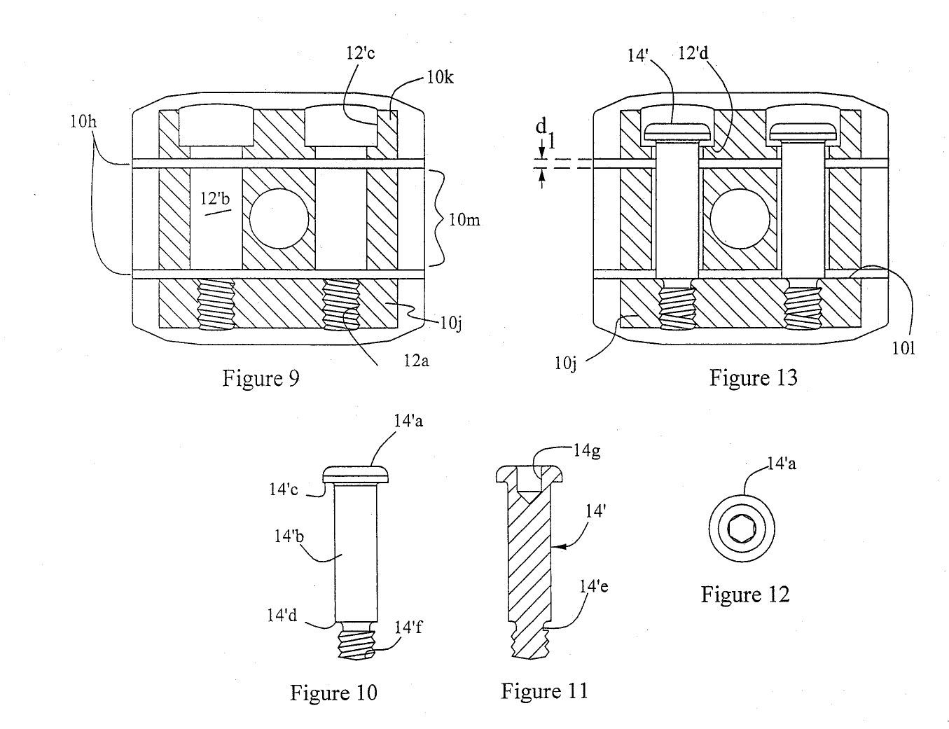

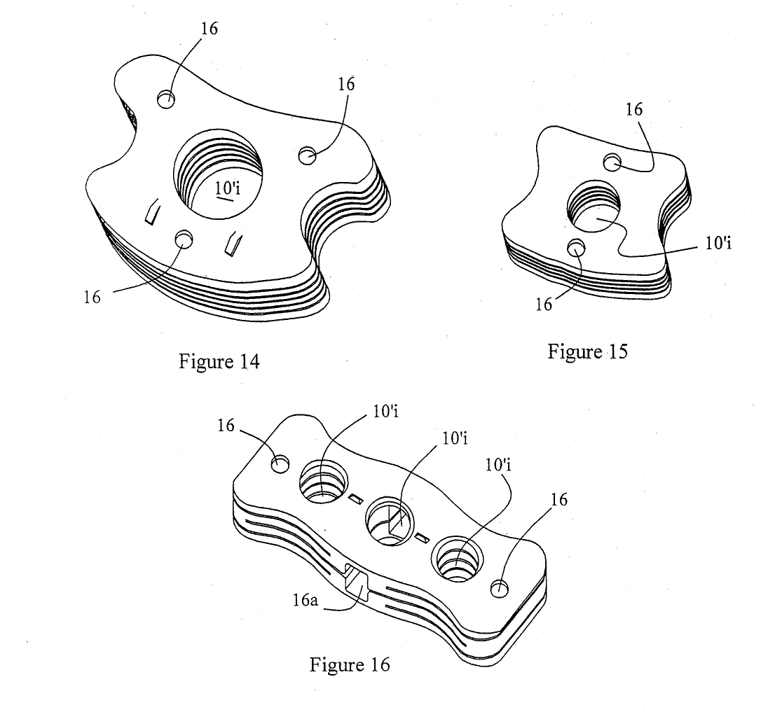

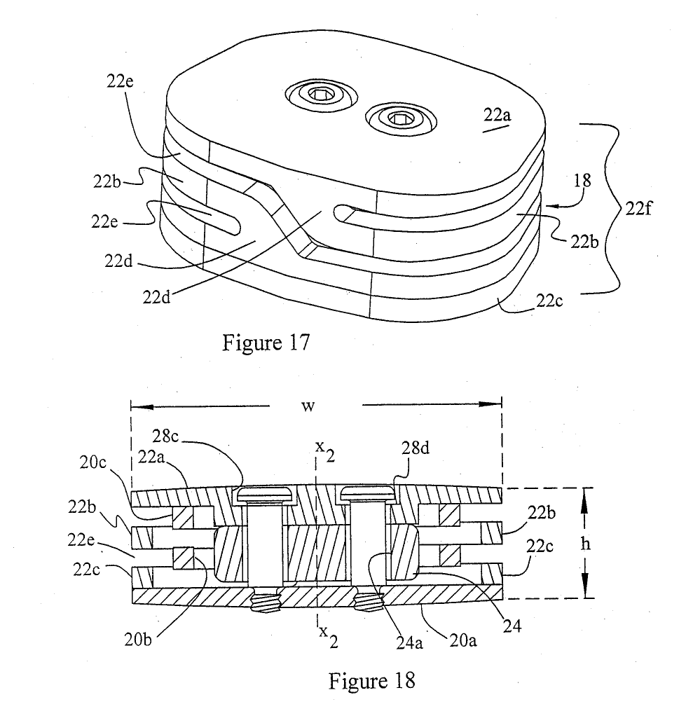

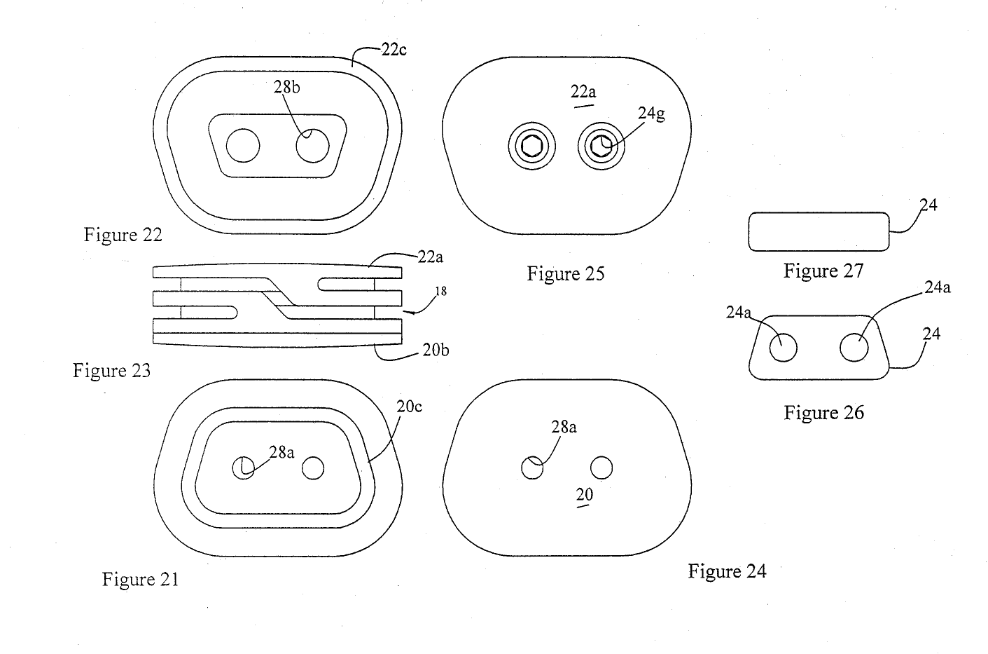

FIGS. 17-26 provide another example of an elastically deformable intervertebral disc 18 in which generally spiral slots in an inner (20) and an outer (22) member form approximately two single hoop spring members for supporting the separated vertebrae while allowing limited compression, expansion and flexion. The disc is arranged around a longitudinal axis x2-x2, as shown.

The outer spring member 22 includes a top plate 22a with a depending spiral spring arm 22b (completing about one turn) joining the top plate to a bottom ring 22c through bridged portions 22d. The spring element is defined by a spiral slot 22e extending through the peripheral wall 22f to form the spring arm 22b as shown.

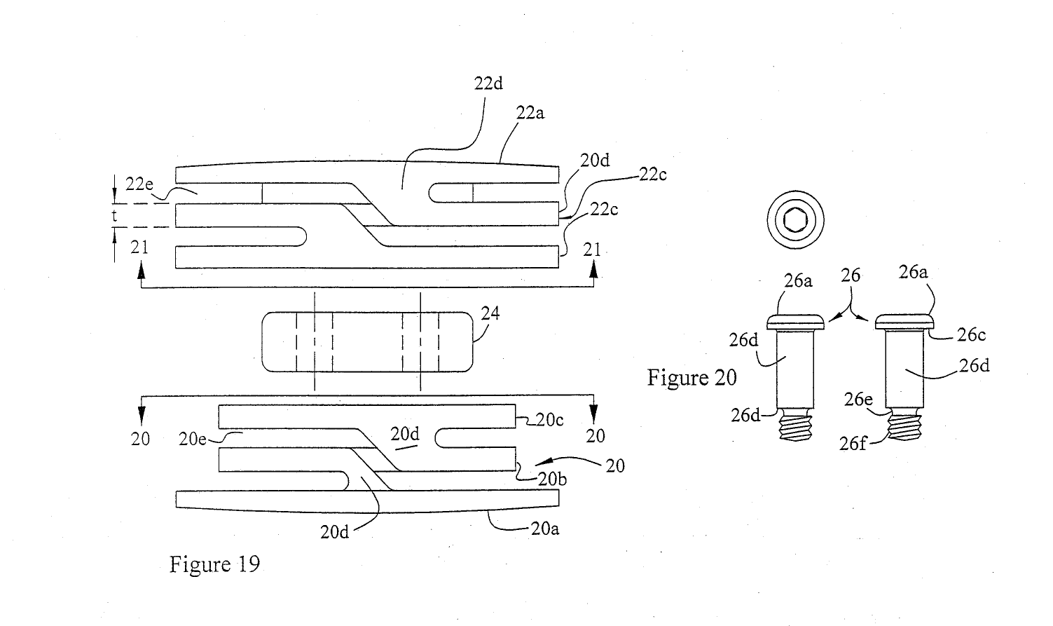

The inner spring member 20 includes a bottom plate 20a with an upwardly extending spiral spring arm 20b (completing about one turn) joining the plate 20a to a top ring 20c through bridge portions 20d. See FIG. 19. Like the spring member 22, the spiral slot 20e forms the spring arm 20b. It is to be noted that the spring member may optionally extend through 1½ to 2 turns. The plates are formed in an oval shape, as shown, to conform to the general shape of the vertebral bodies to be supported.

A centrally located compression limiting spacer 24 is provided with two parallel longitudinal bores 24a for receiving expansion limiting pins 26. The pins have features which are identical to the features of the pins 14 discussed earlier with corresponding features accordingly marked. By the same token, the pin receiving bores 28 in the top and bottom plates are identical to the bore 12 discussed earlier and like features are given the same letter designations.

As an example, an anterior disc, in accordance with FIGS. 17-26, may have a height h (FIG. 18) of 0.235″, a width w across the mid-section (FIG. 18) of 0.520″, and the approximately uniform cross-sectional thickness of the spring arms may be about ______. The extension limit pins are designed to allow about a 0.010″ expansion of the disc from its unstressed state. The compression limiting spacer 24 is designed to allow the same amount of compression.

The device in FIGS. 17-26 may optionally consist of only one spiral slot and in which case the compression limiting spacer 24 would not be required but can be incorporated into the member without a spring. At least one limiting pin 26 would be required to couple the top plate 22a and bottom plate 20a in at least one and up to six degrees of freedom.

As an option, the disc may be formed with a central bone growth accommodating opening extending along the longitudinal axis. It is to be noted, that disc of FIGS. 17-26 represents an improvement over the prior art even without the addition of fatigue protection pins 26. The disc may be manufactured, e.g., machined, from a high strength bio-compatible material, such as stainless steel, titanium or a titanium alloy. The disc may further contain polymers such as polyetheretherketone (PEEK) for improved wear proprieties or other mechanical characteristics. The extension pins may also be made from some polymers and fabrics which may solely resist extension and may be in the form of cables.

Additionally, the limiting pins may be hollow in order to facilitate some fluid transport within the scope of the '541 application. In which case the pin may not extend all the way thru both members but can be captured within the body of the device.

A novel fatigue protection arrangement for intervertebral prosthetic devices has been described. Modifications to the system may occur to those skilled in the art without involving any departure from the spirit and scope of the invention as defined in the appended claims.

Those skilled in the art will appreciate that various adaptations and modifications of the just-described preferred embodiment can be configured without departing from the scope and spirit of the invention. Therefore, it is to be understood that, within the scope of the amended claims, the invention may be practiced other than as specifically described herein.

What is claimed is:

1. An intervertebral prosthesis comprising:
a) an elastically deformable disc arranged around an imaginary longitudinal or vertical axis with upper and lower surfaces, oriented generally perpendicular to the longitudinal axis, for engaging the faces of adjacent vertebral bodies between which a natural disc has been wholly or partially removed and an exterior wall, the disc being arranged to undergo flexion, compression and expansion in response to forces applied by the supported vertebrae to the disc; and
b) at least one pin extending through the disc for limiting the expansion of the disc.

2. The prosthesis of claim 1 wherein the disc defines one or more horizontally oriented slots extending through portions of the exterior wall, the depths of the slots decreasing and increasing during the compression and expansion of the disc, respectively.

3. The prosthesis of claim 1 wherein the vertebral body engaging surfaces are formed on the exterior surfaces of a top and bottom plate, the plates being separated by an outer helical spring.

4. The prosthesis of claim 3 wherein the disc further includes an inner helical spring disposed between the top and bottom plates.

5. The prosthesis of claim 4 wherein each of the helical springs constitutes about 1 to 1½ turns.

6. The prosthesis of claim 4 further including a central member disposed within the inner spring to limit the compression of the disc.

7. An intervertebral prosthesis comprising:
a) an elastically deformable disc arranged around an imaginary longitudinal axis with upper and lower surfaces, oriented generally perpendicular to the longitudinal axis, for engaging the faces of adjacent vertebral bodies between which a natural disc has been wholly or partially removed, and an exterior wall, the disc including a plurality of interleaved slots with alternate slots extending partially through the disc from opposing sides thereof, the slots being oriented in a direction generally parallel to the longitudinal axis, the depths of the slots decreasing and increasing during the compression and expansion of the overlying portion of the disc; and
b) at least one pin extending through the disc for limiting the expansion of the disc.

8. The prosthesis of claim 7 wherein the disc defines one or more vertically oriented bone accommodating channels.

9. The prosthesis of claim 8 wherein the disc includes upper and lower sections separated from each other by the slots.

10. The prosthesis of claim 9 wherein the disc is formed with a nose and a tail section and side sections and wherein at least one of the interleaved slots extends through the nose section and side sections, but not the tail section and at least one of the interleaved slots extends through the tail and side section, but not the nose section.

11. An intervertebral artificial disc arranged around an imaginary vertical axis comprising:
a) a top and bottom plate having generally horizontal outside surfaces adapted to engage the faces of the vertebral bodies to be supported; and
b) an outer and inner helical spring arm interposed between the plates, each spring arm forming about one to two turns.

12. The disc of claim 11 wherein the outer and inner spring arms are formed integrally with the top and bottom plates, respectively.

13. The disc of claim 12 wherein each spring arm forms only about one complete turn.

14. The disc of claim 13 further including at least one extension limit pin extending between the plates to limit the extension of the disc.

15. The disc of claim 14 further including a compression limiting spacer positioned within the inner spring arm.

16. An artificial spinal disc, aligned along an imaginary vertical axis, for supporting adjacent vertebral bodies in their natural spaced relationship while allowing limited motion therebetween comprising:
a) an upper, lower and center section with one or more generally horizontal slits extending between the upper and lower sections and within the center section, the depth of the slits decreasing and increasing during the compression and expansion of the disc, respectively; and
b) at least one longitudinally oriented pin extending between the upper and lower sections for limiting the expansion of the disc.

17. The disc of claim 16 wherein the disc further defines one or more vertically oriented bone channels extending through the upper and lower sections for accommodating bone growth thereon.

18. An intervertebral prosthesis comprising:
a) an interbody disc arranged around an imaginary longitudinal axis having an upper and a lower section, the sections having exterior faces generally perpendicular to the longitudinal axis for engaging the faces of adjacent vertebral bodies between which a natural disc has been wholly or partially removed, the sections being separated by an elastically deformable center section for accommodating the elastic compression/expansion of the disc; and
b) at least one pin extending through the center section and secured to the upper and lower sections for limiting the expansion of the disc while allowing compression thereof.
